(19) United States
(12) Patent Application Publication
(10) Pub. No.: US 2026/0092333 A1
(43) Pub. Date: Apr. 2, 2026

(54) METHOD FOR EVALUATING SEEDLING PLANT CHARACTER OF TRANSGENIC POPLAR AND USE OF OVEREXPRESSED PAGEXPA1 GENE IN PROMOTING XYLEM DEVELOPMENT OF POPLAR

(71) Applicant: Research Institute of Forestry, Chinese Academy of Forestry, Beijing (CN)

(72) Inventors: Fang Tang, Beijing (CN); Xuejiao He, Beijing (CN); Shutang Zhao, Beijing (CN); Yingli Liu, Beijing (CN)

(73) Assignee: Research Institute of Forestry, Chinese Academy of Forestry, Beijing (CN)

(21) Appl. No.: 18/954,995

(22) Filed: Nov. 21, 2024

(30) Foreign Application Priority Data
Sep. 29, 2024 (CN) 2024113711700

(51) Int. Cl. C12Q 1/6895; G01N 1/30; G01N 33/00

(52) U.S. Cl. CPC C12Q 1/6895; G01N 1/30; G01N 33/0098; C12Q 2600/13; C12Q 2600/158; G01N 2001/302

(57) ABSTRACT

The present disclosure provides a method for evaluating a seedling plant character of a transgenic poplar and use of an overexpressed pagEXPA1 gene in promoting xylem development of the poplar, relating to the technical field of forest wood character determination. The method for evaluating the seedling plant character of the transgenic poplar in the present disclosure includes the following steps: S1. detecting whether or not a foreign gene is integrated into a poplar genome through PCR; S2. detecting the expression level of the foreign gene in the poplar; S3. conducting a microstructural analysis on stem vascular tissues of the transgenic poplar; and S4. separating fibers, ducts, and sieve-tube cells through fiber isolation, and staining the isolated fibers to observe the morphology and structure of the fibers, ducts, and sieve-tube cells.

### INCORPORATION BY REFERENCE

This application contains a Sequence Listing which has been submitted electronically in XML format and is hereby incorporated by reference in its entirety. The name of the text file containing the sequence listing is “sequence list_filed.xml”, has a file size of 8,520 bytes, and was created on Nov. 20, 2024.

This application claims priority to China Application No. 2024113711700 filed 29 Sep. 2024, the contents of which are hereby incorporated by reference in their entirety.

### TECHNICAL FIELD

The present disclosure relates to the technical field of forest wood character determination, and in particular, to a method for evaluating a seedling plant character of a transgenic poplar and use of an overexpressed pagEXPA1 gene in promoting xylem development of the poplar.

### BACKGROUND

Transgenic poplar plants refer to poplar individuals that introduce foreign genes or vectors into poplar plants through the genetic engineering technology, and grow and develop in their seedling stages. The transgenic poplar plants show some characteristics that are different from non-transgenic poplars in the early growth stage in terms of growth rate, morphological characteristics, physiological and biochemical characteristics, stress resistance or resistance to pests and diseases. However, direct measurement of wood characters (such as density, strength, etc.) usually needs to be carried out after the trees have grown to a certain age. High-quality, fast-growing transgenic poplars require identification and analysis for the wood characters in the early growth stage, in order to ensure that their fast-growing, high-quality characteristics are reflected and meet subsequent application needs, and to achieve selection of early superior strains. Therefore, there is a need to develop a method capable of identifying the wood character of the poplar in the early growth stage.

Expansin is the main factor that induces cell wall extension. As a plant cell wall protein, the Expansin can loosen plant cell wall components and increase cell wall flexibility. A research shows that the Expansin family plays an important role in plant root extension, fruit ripening, petiole shedding, plant development, stress resistance and the like. At present, the research on the poplar mostly focuses on root growth and elongation, plant morphological development and stress resistance, etc. However, fiber elongation and xylem development are important biological processes in the formation and development of forest wood. Therefore, whether or not the Expansin can affect the development and changes of fiber cells and xylem cells through the extension of cell walls still requires further research and perfection.

### SUMMARY

An objective of the present disclosure is to provide a method for evaluating a seedling plant character of a transgenic poplar and use of an overexpressed pagEXPA1 gene in promoting xylem development of the poplar, so as to solve the problem that the wood character of the poplar cannot be identified and analyzed in the early growth stage in the prior art.

In order to achieve the above-mentioned objective of the present disclosure, the present disclosure provides the following technical solution:

The present disclosure provides a method for evaluating a seedling plant character of a transgenic poplar, including the following steps:
S1. detecting whether or not a foreign gene is integrated into a poplar genome through PCR;
S2. detecting the expression level of the foreign gene in the poplar;
S3. conducting a microstructural analysis on stem vascular tissues of the transgenic poplar; and
S4. separating fibers, ducts, and sieve-tube cells through fiber isolation, and staining the isolated fibers to observe the morphology and structure of the fibers, ducts, and sieve-tube cells.

Preferably, the microstructure analysis includes the following steps:
(1) cutting a poplar stem segment and then slicing the stem segment, with a thickness of 55-65 μm;
(2) mixing the slices with a dye for being stained;
(3) taking the stained slices out of the dye to observe the vascular structure and tissue characteristics of the poplar stem under a microscope; and
(4) measuring and counting the cross-section diameter, xylem area width and xylem cell layer number of transgenic and non-transgenic poplar plants to identify the impact of the foreign gene on the development of the xylem area of the poplar plant.

Preferably, the dye includes a toluidine blue solution or a phloroglucinol dye.

Preferably, the toluidine blue solution has a concentration of 0.8-1.2% w/v, and dyeing time of 8-12 s.

Preferably, the phloroglucinol dye includes a phloroglucinol solution and a hydrochloric acid solution, the phloroglucinol solution has a concentration of 2-4%, and the hydrochloric acid solution has a concentration of 30-40%; and a volume ratio of the phloroglucinol solution to the hydrochloric acid solution is 1.5-2.5:1.

Preferably, the phloroglucinol dye has the dyeing time of 28-32 s.

Preferably, the steps of fiber isolation are as follows:
(1) cutting the poplar xylem tissue into strip samples with a thickness of 0.4-0.6 mm, and then mixing the samples with a maceration solution to obtain a mixture; and
(2) reacting the mixture for 72-96 h under heating conditions until white fibrous tissues appear in the thin strips.

Preferably, the maceration solution includes hydrogen peroxide and glacial acetic acid; the hydrogen peroxide has a concentration of 25-35%; and a volume ratio of the hydrogen peroxide to the glacial acetic acid is 1:2-3.

Preferably, the heating method is metal bath heating, and the heating temperature is 60-70° C.

The present disclosure also provides use of an overexpressed pagEXPA1 gene in promoting development of fiber cells and xylem cells of the poplar.

By adopting the above technical solution, the present disclosure has the following beneficial effects: the method for evaluating the seedling plant character of the transgenic poplar in the present disclosure includes the following steps: S1. detecting whether or not a foreign gene is integrated into a poplar genome through PCR; S2. detecting the expression level of the foreign gene in the poplar; S3. conducting a microstructural analysis on stem vascular tissues of the transgenic poplar; and S4. separating fibers, ducts, and sieve-tube cells through fiber isolation, and staining the isolated fibers to observe the morphology and structure of the fibers, ducts, and sieve-tube cells. Using the method of the present disclosure may comprehensively evaluate the wood character of poplar in an early growth stage. The present disclosure also provides the use of the overexpressed pagEXPA1 gene in promoting the xylem development of the poplar, thus laying the foundation for the improvement of poplar planting resources.

### BRIEF DESCRIPTION OF DRAWINGS

FIG. 1 shows an identification electrophoretogram of an PagEXPA1 overexpressed transgenic plant; where M: Marker; P: Positive control, PagEXPA1 gene overexpression vector plasmid; CK−: negative control, non-transgenic 84K poplar; 1-7: detected OE:PagEXPA1 transgenic positive plants.

FIG. 2 shows the expression level detection result of a target gene in PagEXPA1 overexpressed transgenic plants; where CK: non-transgenic control plants; OE-1˜OE-3: OE: EXPA1-1˜OE:EXPA1-3, positive PagEXPA1 overexpressed transgenic plants.

FIG. 3 shows a vascular structure of the 15th internode of overexpressed PagEXPA1 transgenic and non-transgenic 84K poplar; where A is the vascular tissue of the 15th internode of three PagEXPA1 overexpressed transgenic poplar plants and a non-transgenic 84K poplar; B is a cross-section diameter of the 15^{th} internode of PagEXPA1 overexpressed transgenic poplar plants and a non-transgenic 84K poplar; C is a xylem width (μm); and D is the statistics of xylem cell layers. **P-value 0.01.

FIG. 4 shows a vascular structure of phloroglucinol staining of the 15^{th} internode of PagEXPA1 overexpressed transgenic and non-transgenic 84K poplar plants, where both A and B are vascular tissues of the 15^{th} internode of a non-transgenic 84K poplar, A: an objective lens magnifies 10 times, B: the objective lens magnifies 20 times; C-H are vascular tissues of the 15^{th} internode of three PagEXPA1 overexpressed poplar plants, B, E, G: the objective lens magnifies 10 times, and C, F, H: the objective lens magnifies 20 times. C and D: OE:EXPA1-1(OE-1); E and F: OE:EXPA1-2 (OE-2); G and H:OE: EXPA1-3 (OE-3).

FIG. 5 shows fiber cells and length statistics of the xylem in the 20^{th} internode of PagEXPA1 overexpressed transgenic and non-transgenic 84K poplar, where, A: xylem fiber cells in the 20^{th} internode of the non-transgenic 84K poplar, B-D: the xylem fiber cells of the 20^{th} internode of different lines OE:EXPA1-1 (OE-1), OE:EXPA1-2 (OE-2), and OE:EXPA1-3 (OE-3) of the PagEXPA1 overexpressed transgenic poplar; and E: statistics on plant fiber lengths of non-transgenic 84K poplar and OE-1˜OE-3 transgenic poplar.

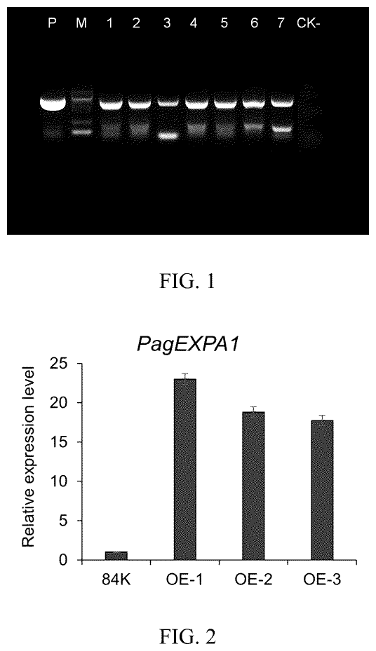

### DETAILED DESCRIPTION

The present disclosure provides a method for evaluating a seedling plant character of a transgenic poplar, including the following steps:
S1. detecting whether or not a foreign gene is integrated into a poplar genome through PCR;
S2. detecting the expression level of the foreign gene in the poplar;
S3. conducting a microstructural analysis on stem vascular tissues of the transgenic poplar; and
S4. separating fibers, ducts, and sieve-tube cells through fiber isolation, and staining the isolated fibers to observe the morphology and structure of the fibers, ducts, and sieve-tube cells.

The microstructure analysis includes the following steps:
(1) cutting a poplar stem segment and then slicing the stem segment, with a thickness of 55-65 μm;
(2) mixing the slices with a dye for being stained;
(3) taking the stained slices out of the dye to observe the vascular structure and tissue characteristics of the poplar stem under a microscope; and
(4) measuring and counting the cross-section diameter, xylem area width and xylem cell layer number of transgenic and non-transgenic poplar plants to identify the impact of the foreign gene on the development of the xylem area of the poplar plant.

In the present disclosure, the stem segment of the poplar is first intercepted, and the stem segment is preferably between the 14th internode and 16th internode, and more preferably the 15th internode. The calculation method of the internodes is: calculating the internode from top to bottom with the stem segment below the first unfolded leaf at the top of the poplar plant as the first internode; the length of the stem segment is 1.5-2.5 cm, further preferably 1.8-2.3 cm, and more preferably 2 cm. The poplar stem segment is then oscillated and sliced, preferably, the poplar stem segment is oscillated and sliced after being fixed with a FAA fixative; the FAA fixative used includes glacial acetic acid and absolute ethyl alcohol, a volume ratio of the glacial acetic acid to the absolute ethyl alcohol is 1:2-4, further preferably 1:2.5-3.5, more preferably 1:3. After being fixed with the FAA fixative, the stem segment is washed 2-3 times and then sliced, and the water used for cleaning is preferably sterilized deionized water. The oscillation and slicing are preferably performed on an oscillating microtome. Before being sliced, the stem segment is cut into approximately 1 cm long, after both ends of the stem segment are trimmed, the stem segment is stuck onto a sample stage of the oscillating microtome, a position between a blade and the sample is adjusted, and the oscillation and slicing are performed after the stem segment sample is fixed. The slicing thickness is 55-65 μm, further preferably 58-62 μm, and more preferably 60 μm. The cut stem segment slices are then transferred to the sterilized deionized water with tweezers for storage or directly stained for observation.

The staining includes toluidine blue staining and phloroglucinol staining. Toluidine blue (TBO for short) can distinguish different vascular tissue cells, and acidic substances in the cells can combine with cations in the TBO dye to be stained. In plant cells, suberized tissues and lignified cell walls stain appear blue-green due to being stained; non-lignified (such as polysaccharide nature) cell walls appear reddish-purple; and proteins and protein-containing mixtures are stained to blue or bluish-violet. The steps for toluidine blue staining are: soaking the cut slice tissues in a toluidine blue solution, the concentration of the toluidine blue solution is 0.8-1.2% w/v, further preferably 0.9-1.1% w/v, and more preferably 1% w/v; and the dyeing time is 8-12 s, further preferably 9-11 s, more preferably 10 s. After the staining is completed, a pipette is used to suck away the toluidine blue solution, and then the slice tissues are rinsed 1-2 times with the sterilized deionized water preferably, and then the vascular structure and tissue characteristics of poplar stems are observed under a microscope. Next, imageJ software (https://github.com/imagej) is used to measure and count the cross-section diameter, xylem area width and xylem cell layer number of PagEXPA1 overexpressed transgenic and non-transgenic poplar plants, to identify the impact of the target gene on the development of the xylem area of the plant.

Phloroglucinol is a lignin dye that can specifically bind to the lignin in the cell wall in an acidic environment to produce a cherry red or purple red reaction, which can be used to determine lignified cell walls. The phloroglucinol dye includes a phloroglucinol solution and a hydrochloric acid solution, and the phloroglucinol solution has a concentration of 2-4%, further preferably 2.5-3.5%, and more preferably 3%; the hydrochloric acid solution has a concentration of 30-40%, further preferably 32-38%, more preferably 37%; and a volume ratio of the phloroglucinol to the hydrochloric acid is 1.5-2.5:1, further preferably 1.8-2.2:1, and more preferably 2:1. The cut slice tissue is soaked in the phloroglucinol dye for being stained, the staining time is 28-32 s, further preferably 29-31 s, and more preferably 30 s; and then after the slice tissue is cleaned 2-3 times, the lignin content of the cell walls in the xylem area of the poplar stem segment is observed under the microscope.

The present disclosure also includes the steps of separating fibers, ducts, and sieve-tube cells through fiber isolation to observe the morphology and structure of the fibers, ducts, and sieve-tube cells. The specific operations of the fiber isolation are as follows: the stem segment of the poplar is intercepted, and the stem segment is preferably between the 19^{th} internode and 21^{th} internode, and more preferably the 20^{th} internode. The poplar xylem tissues are cut into strip samples with a thickness of 0.4-0.6 mm, and the thickness of the strip samples is further preferably 0.45-0.55 mm, more preferably 0.5 mm; then, the strip samples are mixed with the maceration solution, and react for 72-96 h under heating conditions, further preferably 80-90 h, and more preferably 85 h until white fibrous tissues appear in the thin strips. The maceration solution includes hydrogen peroxide and glacial acetic acid; the hydrogen peroxide has a concentration of 25-35%, further preferably 27-32%, and more preferably 30%; and a volume ratio of the hydrogen peroxide to the glacial acetic acid is 1:2-3, further preferably 1:2.2-2.8, and more preferably 1:2.5. The heating method is metal bath heating, and the heating temperature is 60-70° C., further preferably 62-68° C., and more preferably 65° C.

The isolated fibers are then mixed with the toluidine blue solution for being stained. The concentration of the toluidine blue solution is 0.4-0.6%, more preferably 0.5%, and the staining time is 28-32 s, further preferably 29-31 s, and more preferably 30 s. After being stained, the isolated fiber is washed 2-3 times with the sterilized deionized water preferably, and then the stained fiber is placed under a microscope for being observed.

The technical solutions provided by the present disclosure will be described in detail below with reference to the examples, but they should not be understood as limiting the protection scope of the present disclosure.

### Example 1: Acquisition of Transgenic Plants and Detection of Expression Levels

### 1. Transformation of PagEXPA1 Overexpressed Transgenic Plants

Gateway cloning primers were designed based on a sequence of Populus trichocarpa Expansin (Potri.001G240900), as shown in Table 1.

TABLE 1
Sequence of the gateway cloning primers

| Primer | | Serial |
|---|---|---|
| name | Primer sequence | number |
| EXPA1- | 5′-GGGGACAAGTTTGTACAAAA | SEQ ID |
| F | AAGCAGGCTCGATGGCAATGAGC | NO. 2 |
| | AGTTTAATTT-3′ | |
| EXPA1- | 5′-GGGGACCACTTTGTACAAGA | SEQ ID |
| R | AAGCTGGGTCTTAGACCCTGAAA | NO. 3 |
| | TTCTTGCCG-3′ | |

cDNA of Populs alba x P. glandulosa (84K poplar) served as a template, a transcript sequence of PagEXPA1 was obtained by PCR cloning, and the cloned products were ligated to an intermediate vector pDNOR207 using a Gateway™ BP Clonase™ II Enzyme mix kit (#11789020, Invitrogen) according to the instructions, then the vector plasmid was sequenced and the cloned products were determined to be ligated to the intermediate vector, and the intermediate vector was ligated to an overexpressed vector pMDC32 using multiple cloning site through a Gateway™ LR Clonase™ II Enzyme mix kit (#11791100, Invitrogen), to construct the PagEXPA1 overexpressed vector. The constructed PagEXPA1 overexpressed vector was introduced into the 84K polar through Agrobacterium-mediated leaf disc method, and resistant poplar tissue culture seedlings were obtained through resistance screening (hygromycin resistance).

The CDS sequence of the PagEXPA1 gene is shown
in SEQ ID NO. 1:
ATGGCAATGAGCAGTTTAATTTGCATTGCCACTAGTTTACTAATA
ATAGTGTCATCGTTGTGGATGGCTAAAGCTAGAATTCCTGGTGTTTAC
TCCGGGGGTGCTTGGGAAAATGCTCATGCAACCTTCTATGGCGGTTC
TGATGCCTCTGGCACAATGGGAGGAGCTTGTGGATATGGAAATCTGT
ACAGCCAAGGGTATGGAGTGAGCACTGCAGCCCTAAGCACAGCACT
GTTCAACAACGGGTTAAGTTGCGGTTCTTGCTTCGAGATAAAATGTG
CAAGTGACCCGAGATGGTGCCACTCAGGCAGCCCGTCTATTTTCATC
ACTGCAACCAACTTTTGCCCTCCAAATTATGCACTTCCTAGTGACAAT
GGAGGCTGGTGCAACCCTCCTCGCCCCCACTTTGACCTTGCCATGCCC
ATGTTCCTTAAGATCGCCGAGTATCGTGCCGGTATCGTCCCTGTTGCC
TACCGCCGAGTGCCATGCCGCAAGAGGGGAGGTATAAGGTTCACTAT
AAAACGGATTCCGTTACTTCAACTTGGTATTGATCAGCAACGTGGCG
GGTGCAGGGGATATAGTGCAGGTGAGCGTGAAGGGTTCAAAGACTG
GTTGGATGAGCATGAGCCGTAACTGGGGCCAGAACTGGCAGTCAAA
CGCTGTTCTGGTTGGCCAGACACTCTCCTTCAGGGTTAGGGCCAGTG
ACAGACGGCTCCTCCACTTCATGGAACATTGTCCCAGCCCACTGGCA
GTTTGGTCCAAACTTTTACCGGCAAGAATTTCAGGGGGAGGGAGACT
AAGGCTGAGGCGGCTGCACAAAAGATGTTGAGGGGCCATGTACGAG
AGGTCATTGCACGCAGGGCAAAACCAAATTGACTTTCAAAGCTTGAG
GGCTAAAAAAGTTGTTGTGGAGGCTAAGCTAATGGACCTCAAGATAG
CCTATGCAACACTAGAGAATATCATGAAAAGCCAAGTCCTTCAGCTA
AACTCTAGTGAAAGGGAGCTTCGTCTTGCTTATGATAAAATGGTCTTT
CTAAAGCAAGGGATGGTTGAGATGAGGGAATTCTAG.

2. Detection Whether or not the Foreign Gene has been Integrated into the Poplar Genome Through PCR

The leaf tissues of the hygromycin-resistant 84K poplar and non-transgenic 84K poplar tissue culture seedlings were used as materials, and their genomic DNAs were extracted for PCR molecular detection in respective. Because the overexpressed vector contains the resistance gene hygromycin phosphotransferase gene HptII, the upstream primer HYP-F: 5′ CGTCTCCGACCTGATGCAGCTCT-3′ (SEQ ID NO.4) and the downstream primer HYP-R: 5′-ATCCTGCAAGCTCCGGATGCCTC-3′ (SEQ ID NO.5) were designed according to the sequence of this gene for PCR amplification. If the result has an amplified band and the size is about 700 bp, it can be used as a sign of successful transgene. The non-transgenic 84K tissue culture seedlings served as a negative control and the overexpressed vector plasmid served as a positive control, to perform the PCR detection on the obtained resistant tissue culture seedlings.

The 50 μL PCR reaction system was as follows: 0.25 μL Taq DNA polymerase (5 U/μL), 5 μL 10×PCR buffer (containing Mg²⁺), 4 μL dNTP Mixture (2.5 mM each), 2 μL Primer (10 μM), and 500 ng plant DNA were complemented to 50 μL with sterilized deionized water. A reaction tube was placed into a PCR instrument and the PCR amplification program was set according to the following parameters: 95° C. for 5 min, 95° C. for 20 s, 58° C. for 30 s, 72° C. for 30 s, 35 cycles, 72° C. for 7 min, and stored at a constant temperature of 4° C. Each tissue culture seedling that could detect a band of the same size as the positive control (about 700 bp) was propagated as a positive transgenic line; while the tissue culture seedlings in which the target fragments were not detected were false positive or non-transgenic lines. The results showed that there were 7 positive transgenic lines, from which 3 lines (OE:EXPA1-1, OE:EXPA1-2, OE:EXPA1-3) were randomly selected for subsequent functional studies.

### 3. Detection of PagEXPA1 Expression Level in Transgenic Plants

Leaf tissues of the tissue culture seedlings of three lines (OE:EXPA1-1, OE:EXPA1-2, OE:EXPA1-3) of PagEXPA1 transgenic positive 84K poplar and non-transgenic 84K poplar were used as materials, and their total RNA was separately extracted using RNAprep Pure Plant Plus kit (#DP441, TianGen, China). The integrity of total RNA was assessed by 1.5% agarose gel electrophoresis, and RNA concentration and purity were determined by NanoDrop™ 8000 spectrophotometer. Only RNA samples with an A260/A280 ratio between 1.9 and 2.1 and an A260/A230 ratio greater than 1.80 were used for cDNA synthesis. PrimeScript™ RT reagent Kit (#RR037, TaKaRa, Japan) was used to perform reverse transcription synthesis of cDNA on 1 μg of qualified RNA sample. The quantitative amplification primer qEXP-F: 5′-GAGCAGTTTAATTTGCATTGCCACT-3′ (SEQ ID NO. 6) and qEXP-R: 5′-AGAGTAAACACCAGGAATTCTAGCTT-3′ (SEQ ID NO. 7) of the PagEXPA1 gene were used to detect the relative expression level of the target PagEXPA1 gene in a LightCycler® 480 System quantitative PCR instrument (Roche, Germany). The 20 μL PCR reaction system was as follows: 10 μL KAPA SYBR FAST qPCR Master Mix (#K4601, KAPA Biosystems, USA), 2 μL 20-fold diluted cDNA, 0.4 μM forward primer (qEXP-F) and 0.4 μM reverse primer (qEXP-R) were complemented to 20 μL with the sterilized deionized water. The quantitative PCR amplification was performed according to the following procedure: 95° C. for 3 s, 95° C. for 10 s, 60° C. for 30 s, 72° C. for 3 s, and 40 cycles. The reaction without template was used as a negative control, and PP2A-2 was used as an internal reference gene. Each sample was evaluated in four technical replicates, and the experiment was repeated 3 times. The relative expression level of the data was calculated using the 2^{-ΔΔCT} method. By comparing the expression levels of the target gene PagEXPA1 in transgenic and non-transgenic plants, the impact of the overexpressed PagEXPA1 gene on the development of fiber cells and xylem cells of the poplar can be evaluated.

### Example 2: A Method for Measuring Wood Character Indicators of Transgenic Plants

### 1. Cultivation and Growth of Transgenic Plants

PagEXPA1 overexpressed transgenic tissue culture seedlings and non-transgenic poplar tissue culture seedlings were subjected to tissue culture, subculture and transferring at the same time, the rooting of the transgenic poplar tissue culture seedlings served as the starting point, after 40 days of growth of the tissue culture seedlings, 3 PagEXPA1 overexpressed transgenic lines and non-transgenic poplar were selected, 4 tissue culture seedlings were selected from each line to be transplanted into the soil at the same time for growing and being cultured in a culture room, and the culture temperature was 25-28° C. and the illumination time was 16 h. After PagEXPA1 overexpressed transgenic and non-transgenic poplar plants were cultured in soil for 80 days, microscopic observation and statistical analysis such as tissue slicing and fiber isolation were performed.

### 2. Microstructural Analysis on Stem Vascular Tissue

Purpose: by using a light microscope or an electron microscope to compare microstructures of the stems of annual transgenic and non-transgenic poplar plants, how the foreign genetic modification affects the cell morphology, tissue structure and arrangement of the poplar can be intuitively observed, which is helpful an in-depth understanding of how the foreign gene affects the growth, development, phenotype and wood properties of the poplar.

Method: generally speaking, the annual poplar has basically no lateral branches, and each leaf or lateral bud represents a node. Therefore, the stem segment below the first unfolded leaf at the top of the poplar plant cultured in soil for 80 days was taken as a first internode. The internodes were counted from top to bottom, and the 15th internode was selected for tissue slicing and microscopic observation.

### (1) Slicing

A single-sided blade was used to cut a stem segment of about 2 cm in the middle of the 15th internode, and the stem segment was fixed with a FAA fixative (glacial acetic acid: absolute ethyl alcohol=1:3) or directly oscillated and sliced. The stem segments fixed with the FAA fixative needed to be washed three times with sterilized deionized water before being sliced. The cross slicing of the stem segment was performed on a Leica VT1000S oscillating microtome. Before slicing, the single-sided blade was used to cut a stem segment about 1 cm in the middle of the sample, and after being trimmed, both ends of the stem segment were stuck to the sample stable of the oscillating microtome with Loctite 495 glue, the position between the blade and the sample was adjusted, after the stem segment sample was fixed, oscillation and slicing were performed, the slicing thickness was 60 μm, and the cut stem segment was sliced for staining and observation.

### (2) Staining

\textcircled{1} Toluidine blue staining: toluidine blue (TBO for short) can distinguish different vascular tissue cells. The acidic substances in the cells can combine with the cations in the TBO dye to be stained. In plant cells, suberized tissue and lignified cell walls appeared blue-green due to being stained; non-lignified (such as polysaccharide nature) cell walls appeared reddish-purple; and proteins and protein-containing mixtures were dyed blue or bluish-violet.

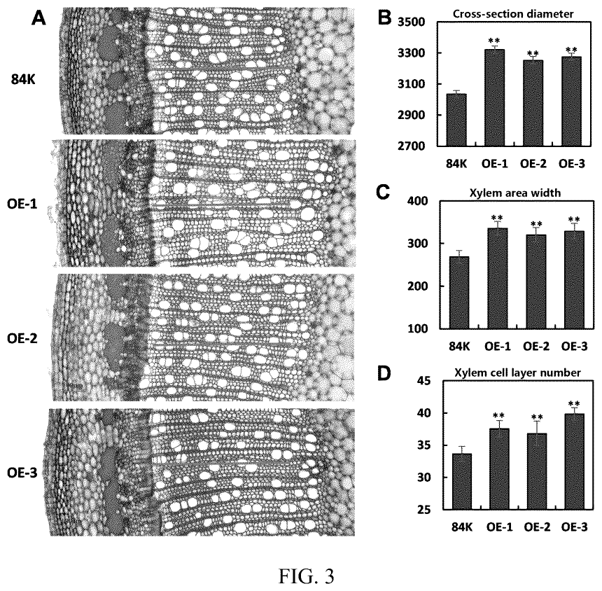

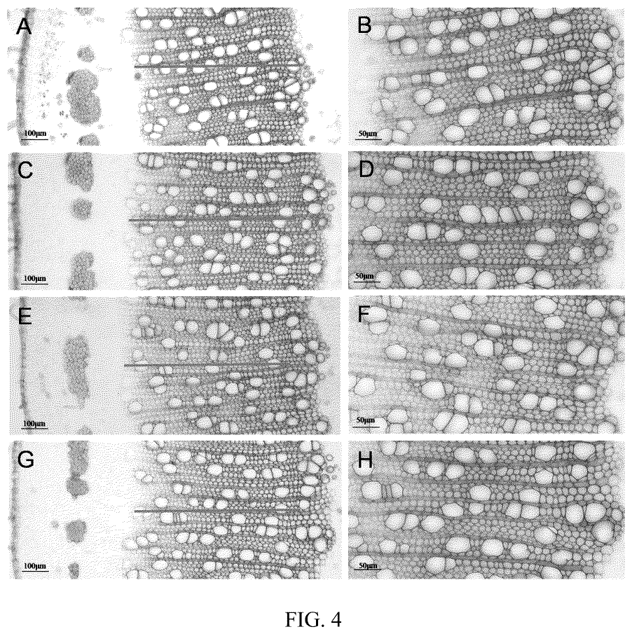

The cut slice tissue was completely soaked in 1% (w/v) TBO solution, the slice was taken out after 10 s, a pipette was used to absorb the excess TBO staining solution, and the slice tissue was washed twice with sterilized deionized water. The vascular structure and tissue characteristics of the stems of transgenic poplar and non-transgenic poplar were observed under the bright field of an Olympus BX51 microscope. ImageJ software (https://github.com/imagej) was used to measure and count the cross-section diameter, xylem area width and xylem cell layer number of PagEXPA1 overexpressed transgenic and non-transgenic poplar plants to identify the impact of the target gene on the development of the xylem area of the plant. The results are shown in FIG. 3. As can be seen from the figure, compared with non-transgenic 84K plants, the cross-section diameter and xylem tissue of plants in different lines overexpressed by PagEXPA1 significantly increased (A in FIG. 3), the cross-section diameters of OE:PagEXPA1-1˜OE:PagEXPA1-3 plants increased by 9.4%, 7.2% and 7.9% respectively (B in FIG. 3), the xylem width increased by 24.8%, 19.2% and 22.3% respectively (C in FIG. 3), and the cell layer number in the xylem area increased by 11.6%, 9.5% and 18.3% respectively (D in FIG. 3). This shows that the thickening of the xylem area of PagEXPA1 overexpressed plants may be caused by the increase in the number of xylem cell layers. This also indicates that changes in PagEXPA1 expression alter the radial growth and xylem development rate of the poplar plants.

\textcircled{2} Phloroglucinol staining: phloroglucinol is a lignin dye that can specifically bind to the lignin in the cell wall in an acidic environment to produce a cherry red or purple red reaction, which can be used to identify lignified cell walls. The phloroglucinol dye is a phloroglucinol-HCL solution formed by mixing 3% phloroglucinol solution and 37% concentrated hydrochloric acid in a volume of 2:1. The tissue slices were put into phloroglucinol-HCl solution for being stained. After about 30 s, the solution was aspirated, and after the tissue slices were washed 3 times with the sterilized deionized water, the lignin content of the cell walls in the transgenic poplar and non-transgenic 84K poplar stem xylem area was observed under a light field of an Olympus BX51 microscope, with the results shown in FIG. 4.

As can be seen from FIG. 4, after phloroglucinol staining, the color of the xylem cell walls of the three lines (C-H in FIG. 4) of the overexpressed PagEXPA1 gene has a little difference from that of the non-transgenic 84K plants (A and B in FIG. 4), but the specific content changes require chemical determination of the lignin content. Thus, it can be seen that this method can be used to predict the lignin content of plants in the early plant growth stage.

### Example 3: Fiber Isolation and Cell Observation of Stem Xylem Cells

Purpose: through fiber isolation, cells such as fibers and ducts can be separated from the cell wall in order to observe the morphology and structure of cells such as fibers, ducts and sieve-tube cell, which can understand the basic information such as the microstructure and chemical composition of wood deeply, and help to understand the physical and mechanical properties of wood, thus providing basic data for further analysis of wood characters.

Method: fiber isolation is carried out using acid treatment method. The maceration solution is prepared by mixing 30% hydrogen peroxide and glacial acetic acid in a volume ratio of 3:7. The 20^{th} stem segment of PagEXPA1 overexpressed transgenic and non-transgenic poplar plants cultured in soil for 80 days was used as material for fiber isolation.

### (1) Fiber Isolation Method

A single-sided blade was used to cut a stem segment of about 2 cm in the middle of the 20^{th} node. The outer bark of the stem segment was removed and the tissue sample within the xylem was kept only, the sample was into thin strips of about 0.5 mm, and then soaked in a 2 mL centrifuge tube containing the maceration solution. The samples in the centrifuge tubes were incubated in a metal bath at 65° C. for 80 h until white fibrous tissue appeared in thin strips. After a short period of centrifugation in a desktop microcentrifuge, a pipette was used to absorb as much of the maceration solution as possible, and then the sample was washed 3 times with the sterilized deionized water (that is, add sterilized water to be mixed well, centrifuge slightly and then absorb the sterilized water), then the sample was stored in the sterilized water.

### (2) Staining Method and Observation Statistics

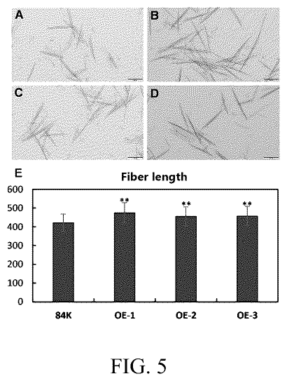

The isolated fibers were transferred to a 2.0 mL centrifuge tube containing 0.5% (w/v) toluidine blue (TBO) solution for being stained for 30 s, and then the isolated fibers were washed 3 times with the sterilized deionized water. The stained fibers can be imaged and measured using an Olympus BX51 microscope. ImageJ software (https://github.com/imagej) was used to measure the fiber length of PagEXPA1 overexpressed transgenic and non-transgenic poplar plants, and 200 fibers were randomly measured for each plant. The measurement results were counted using SPSS software to calculate the average length and variance of the fibers, as well as significant difference analysis, with the results shown in FIG. 5.

As can be seen from FIG. 5, compared with non-transgenic 84K plants, the fiber length of plants of different lines overexpressed by PagEXPA1 increased significantly (A-D in FIG. 5). The fiber length of OE: PagEXPA1-1˜OE:PagEXPA1-3 plants increased by 12.7%, 8.2% and 8.6% respectively (E in FIG. 5), indicating that PagEXPA1 overexpression has promoted the growth of fibers in plant cell walls.

As can be seen from the above examples, the present disclosure provides a method for evaluating a seedling plant character of a transgenic poplar and use of an overexpressed pagEXPA1 gene in promoting xylem development of the poplar. The method for identifying the wood character of transgenic poplar seedling plants of the present disclosure includes multiple aspects such as molecular biological detection and measurement of basic physiological indicators. The molecular biological detection can detect the stability of the foreign gene in the transgenic plants and the impact of the foreign gene on the plant character at the molecular level, and the wood character of poplar in the early growth stage can be comprehensively evaluated through the microstructural analysis of the stem vascular tissue, and fiber isolation and cell observation of the stem xylem cells.

The above are only the preferred embodiments of the present disclosure. It should be pointed out that those of ordinary skill in the art can also make several improvements and modifications without departing from the principles of the present disclosure. These improvements and modifications should be regarded as the protection scope of the present disclosure.

What is claimed is:

1. A method for evaluating a seedling plant character of a transgenic poplar, comprising the following steps:
S1. detecting whether or not a foreign gene is integrated into a poplar genome through PCR;
S2. detecting an expression level of the foreign gene in the poplar;
S3. conducting a microstructural analysis on stem vascular tissues of the transgenic poplar; and
S4. separating fibers, ducts, and sieve-tube cells through fiber isolation, and staining the isolated fibers to observe the morphology and structure of the fibers, ducts, and sieve-tube cells.

2. The method according to claim 1, wherein the microstructural analysis comprises the following steps:
(1) cutting a poplar stem segment and then slicing the stem segment, with a slicing thickness of 55-65 μm;
(2) mixing the slices with a dye for being stained;
(3) taking the stained slices out of the dye to observe the vascular structure and tissue characteristics of the poplar stem under a microscope; and
(4) measuring and counting the cross-section diameter, xylem area width and xylem cell layer number of transgenic and non-transgenic poplar plants to identify the impact of the foreign gene on the development of the xylem area of the poplar plant.

3. The method according to claim 2, wherein the dye comprises a toluidine blue solution or a phloroglucinol dye.

4. The method according to claim 3, wherein the toluidine blue solution has a concentration of 0.8-1.2% w/v, and dyeing time of 8-12 s.

5. The method according to claim 3, wherein the phloroglucinol dye comprises a phloroglucinol solution and a hydrochloric acid solution, the phloroglucinol solution has a concentration of 2-4%, and the hydrochloric acid solution has a concentration of 30-40%; and a volume ratio of the phloroglucinol solution to the hydrochloric acid solution is 1.5-2.5:1.

6. The method according to claim 5, wherein the phloroglucinol dye has the dyeing time of 28-32 s.

7. The method according to claim 1, wherein the steps of the fiber isolation are as follows:
(1) cutting the poplar xylem tissue into strip samples with a thickness of 0.4-0.6 mm, and then mixing the samples with a maceration solution to obtain a mixture; and
(2) reacting the mixture for 72-96 h under heating conditions until white fibrous tissues appear in the thin strips.

8. The method according to claim 7, wherein the maceration solution comprises hydrogen peroxide and glacial acetic acid, the hydrogen peroxide has a concentration of 25-35%, and a volume ratio of the hydrogen peroxide to the glacial acetic acid is 1:2-3.

9. The method according to claim 7, wherein the heating method is metal bath heating, and the heating temperature is 60-70° C.

10. Use of an overexpressed pagEXPA1 gene in promoting xylem development of a poplar.


SEQUENCE LISTING (7 sequences)

SEQ ID NO: 1 - DNA, 1068 bp, synthetic construct
  source 1..1068: mol_type = other DNA
atggcaatgagcagtttaatttgcattgccactagtttactaataatagtgtcatcgttgtggatggctaaagctagaattcctggtgtttactccgggggtgcttgggaaaatgctcatgcaaccttctatggcggttctgatgcctctggcacaatgggaggagcttgtggatatggaaatctgtacagccaagggtatggagtgagcactgcagccctaagcacagcactgttcaacaacgggttaagttgcggttcttgcttcgagataaaatgtgcaagtgacccgagatggtgccactcaggcagcccgtctattttcatcactgcaaccaacttttgccctccaaattatgcacttcctagtgacaatggaggctggtgcaaccctcctcgcccccactttgaccttgccatgcccatgttccttaagatcgccgagtatcgtgccggtatcgtccctgttgcctaccgccgagtgccatgccgcaagaggggaggtataaggttcactataaacggattccgttacttcaacttggtattgatcagcaacgtggcgggtgcaggggatatagtgcaggtgagcgtgaagggttcaaagactggttggatgagcatgagccgtaactggggccagaactggcagtcaaacgctgttctggttggccagacactctccttcagggttagggccagtgacagacgctcctccacttcatggaacattgtcccagcccactggcagtttggtcaaacttttaccggcaagaatttcagggggagggagactaaggctgaggcggctgcacaaaagatgttgagaggggccatgtacgagaggtcattgcacgcagggcaaaaccaaattgactttcaaagcttgagggctaaaaaagttgttgtggaggctaagctaatggacctcaagatagcctatgcaacactagagaatatcatgaaaagccaagtccttcagctaaactctagtgaaagggagcttcgtcttgcttatgataaaatggtctttctaaagcaagggatggttgagatgagggaattctag

SEQ ID NO: 2 - DNA, 53 bp, synthetic construct
  source 1..53: mol_type = other DNA
ggggacaagtttgtacaaaaaagcaggctcgatggcaatgagcagtttaattt

SEQ ID NO: 3 - DNA, 52 bp, synthetic construct
  source 1..52: mol_type = other DNA
ggggaccactttgtacaagaaagctgggtcttagaccctgaaattcttgccg

SEQ ID NO: 4 - DNA, 23 bp, synthetic construct
  source 1..23: mol_type = other DNA
cgtctccgacctgatgcagctct

SEQ ID NO: 5 - DNA, 23 bp, synthetic construct
  source 1..23: mol_type = other DNA
atcctgcaagctccggatgcctc

SEQ ID NO: 6 - DNA, 25 bp, synthetic construct
  source 1..25: mol_type = other DNA
gagcagtttaatttgcattgccact

SEQ ID NO: 7 - DNA, 26 bp, synthetic construct
  source 1..26: mol_type = other DNA
agagtaaacaccaggaattctagctt